(19) United States
(12) Patent Application Publication
(10) Pub. No.: US 2007/0015702 A1
(43) Pub. Date: Jan. 18, 2007

(54) Method of treatment and/or prevention of neuropathic pain

(75) Inventors: Akitoshi Ito, Tokyo (JP); Mineko Takeda, Tokyo (JP)

Correspondence Address: EDWARDS & ANGELL, LLP, P.O. BOX 55874, BOSTON, MA, 02205, US

(73) Assignee: Asahi Kasei Pharma Corporation, Tokyo (JP)

(21) Appl. No.: 11/449,159

(22) Filed: Jun. 7, 2006

Related U.S. Application Data
(60) Provisional application No. 60/689,232, filed on Jun. 10, 2005.

(30) Foreign Application Priority Data
Jun. 9, 2005 (JP) 2005-170165

(51) Int. Cl. A61K 38/23

(52) U.S. Cl. 514/12

(57) ABSTRACT

The present invention relates to a method of treating and/or preventing neuropathic pain, comprising administering calcitonin continuously. The present invention can provide a drug effective for the treatment of various neuropathic pains. Providing a drug having an action effective on neuropathic pain and having low toxicity. An agent for treating and/or preventing neuropathic pain comprising calcitonin, particularly elcatonin, as an active ingredient, wherein calcitonin is administered in an amount not altering serum calcium value, and use of calcitonin, particularly elcatonin, for producing an agent for treating and/or preventing neuropathic pain.

### BACKGROUND OF THE INVENTION

1. Field of the Invention

The present invention relates to a calcitonin preparation having a quick-acting analgesic effect on neuropathic pain.

2. Description of Prior Art

Neuropathic pain is an intractable pain caused by dysfunction in the peripheral or central nervous system. Neuropathic pain is initiated by neuropathy caused by metabolic disorders such as trauma, infection, cancer, ischemia, diabetes and the like. Though the onset mechanism is not very clear, abnormal continuous firing of the sensory nerve and the like are thought to be the causes. Typical symptoms of neuropathic pain include allodynia, hyperalgesia, hyperesthesia and the like. These symptoms manifest characteristic pains described as “burning”, “stinging”, “electroshock-like” and the like. It is known that analgesics effective for usual nociceptive pain, particularly narcotic analgesics, and the like are not so effective for neuropathic pain (The Lancet 353, 1959-1966, 1999). Conventionally known analgesics include central opioid-based analgesics typified by morphine, nonsteroidal anti-inflammatory drugs (NSAIDs) typified by indomethacin, and the like. These drugs, however, have a poor effect on neuropathic pain and, like NSAIDs on acute pain, just blocking the production of an algesic substancecannnor remove the pain.

It is known that while morphine has a strong analgesic action on nociceptive pain, it does not show a sufficient effect on neuropathicpain (IgakunoAyumi, 189(10), 751-755, 1999). The reason that morphine is not effective on neuropathic pain is believed to be due to the decrease of opioid receptors (Saishin Nou to Shinkeikagaku Series, vol. 6, Itami no Shinkeikagaku, Medical View, p97, 1997) caused by the functional and morphological changes in the nerve caused by neuropathy.

Therefore, various factors are believed to be intricately interrelated with the onset of neuropathic pain. Current treatment methods are neurosurgical treatments such as nerve block, spinal epidural electric stimulation and the like (Igaku no Ayumi, 189(10), 757-762), lumbar intraspinal administration of baclofen (Kinoteki Noushinkeikagaku 3345-49, 1994), and the like.

Further, in pain therapy, continuation of pain causes a plastic change in the neural network, shifting to intractable pain. This is considered problematical, thus, early pain control is one of the most important tasks (Kumazawa Takao; Itami ha Hizumu. Nou wo shiru. edited by Hisano Takashi, Shujunsha, 106-116, 1999).

On the other hand, a calcitonin preparation is used for treatment of hypercalcemia and osteoporosis, and acts via a calcitonin receptor of an osteoclast.

Calcitonin receptors on an osteoclast cause down-regulation when calcitonin is allowed to act continuously, which is feared to result in the inability of serum calcium concentration control. Since no disjunction of effective concetration between variation in serum calcium and effective concentration of analgesic action has been reported thus far, a calcitonin preparation is administered intermittently, daily or once per week.

It is known up to now that calcitonin has an analgesic action on a certain kind of pain, and it is also known that its action mechanism differs depending on the kind of pain. For example, for inflammatory pain, a prostaglandin production suppressing action and correlation of intracerebral ascending serotonin nerve system are reported. Regarding pain of bone metastasis cancer, an inhibitory action for a bone resorption at a bone metastasis site and an action via secretion of β-endorphin (endogenous opioid) are known. However, just as NSAIDs and morphine are not very effective, these mechanisms are not expected to present substantially effective action on neuropathic pain. In neuropathic pain caused by peripheral nerve disorders, it is believed that abnormal continuous firing of the sensory nerve, its threshold decrease, and hyperalgesia are occurring. These phenomena are reported to be induced by altered gene expressions such as voltage-dependent sodium channel in the sensory nerve, and believed to be a pathological condition independent of other pains (Boucher T J et al: Potent analgesic effects of GDNF in neuropathic pain states. Science, 2000, 290, 124-127, Hong S et al, Early painful diabetic neuropathy is associated with differential changes in tetrodotoxin-sensitive and resistant sodium channels in dorsal root ganglion neurons in the rat. J Biol Chem. 2004 Jul. 9; 279(28): 29341-50).

There is a clinical report that intermittent administration of a calcitonin preparation is effective on various neuropathic pains, but a longer period of time was necessary for the effect to manifest, and also, its effect is substantially utterly insufficient (Wade S et al: A critical reviews of controlled clinical trials for peripheral neuropathic pain and complex regional pain syndromes, PAIN, USA, 1997, 73, 123-139, Antonio Quatraro, calcitonin in painful diabetic neuropathy, THE LANSET, 1992, 339, 746-747).

As described above, in treatment of neuropathic pain, a safe and quick-acting effective therapeutic method is not established and the development of a new therapy is eagerly desired.

### SUMMARY OF THE INVENTION

The object of the present invention is to provide a novel medicine having an excellent quick-acting analgesic action on neuropathic pain.

For attaining the above-mentioned object, a study has been intensively carried out focusing attention on calcitonin, particularly elcatonin. Though it was difficult to develop a substantially effective neuropathic pain treating drug containing calcitonin as an active ingredient, from an action mechanism of an analgesic action of calcitonin and intermittent administration results-for neuropathic pain known until now, an action of calcitonin for repairing gene expression of Na channel in sensory nerve (Ito et al., Osteoporosis Japan, 2005, vol. 13, no. 1, 78-80) was found. Therefore, optimization of this analgesic action mechanism has been investigated imagining that calcitonin would have a completely separate novel analgesic pharmacological action mechanism. Specifically, a calcitonin administration method which manifests a quick-acting analgesic action on neuropathic pain without altering the serum calcium value has been intensively studied using a model evaluation system for neuropathic pain, and consequently it has been found, surprisingly, that a quick-acting strong analgesic action is shown when calcitonin, particularly elcatonin is administered continuously, further that the same effect is shown also by continuous administration of dosage without altering serum calcium, leading to the present invention based on these findings.

That is, the present invention relates to

(1) An agent for treating and/or preventing neuropathic pain comprising calcitonin as an active ingredient, wherein calcitonin is administered in an amount that does not change the serum calcium value.

(2) An agent for treating and/or preventing neuropathic pain comprising calcitonin as an active ingredient, wherein calcitonin is administered in an amount less than 0.6 mU/kg/second.

(3) The agent for treating and/or preventing neuropathic pain according to (1) or (2), wherein the agent is administered continuously.

(4) The method of treating and/or preventing neuropathic pain according to (3), wherein the dose per unit time (second) of calcitonin in continuous administration is an amount equal to or less than one-hundredth of the dose per unit time (second) of calcitonin in each administration effective for treatment of neuropathic pain in intermittent administration.

(5) The agent for treating and/or preventing neuropathic pain according to (3) or (4), wherein the duration of the continuous administration is 8 hours or more.

(6) The agent for treating and/or preventing neuropathic pain according to any one of (1) to (5), wherein the neuropathic pain is caused by the compression of the peripheral nerve or the injury of the peripheral nerve.

(7) The agent for treating and/or preventing neuropathic pain according to any one of (1) to (6), wherein the calcitonin is elcatonin.

(8) An agent for treating and/or preventing neuropathic pain for continuous administration comprising calcitonin as an active ingredient, wherein the dosage form is any one of sustained release, drip and patch preparations.

(9) The agent for treating and/or preventing neuropathic pain according to (8), wherein the dose per unit time (second) of calcitonin in continuous administration is an amount equal to or less than one-hundredth of the dose per unit time (second) of calcitonin in each administration effective for treatment of neuropathic pain in intermittent administration.

(10) The agent for treating and/or preventing neuropathic pain according to any one of (8) to (9), wherein the duration of continuous administration is 8 hours or more.

(11) Use of calcitonin for producing the agent for treating and/or preventing neuropathic pain according to any one of (1) to (10).

The present invention provides an agent for treating and/or preventing neuropathic pain which can reduce patient load and side effects by drug administration and which is more effective and quick-acting.

### BRIEF EXPLANATION OF DRAWINGS

FIG. 1 shows change in nociceptive threshold in continuous administration of elcatonin against lowering of nociceptive threshold of sciatic nerve constriction model.

**: comparison of nociceptive threshold before and after a constriction operation by t-test p<0.01

#: comparison of nociceptive threshold of a vehicle administered group and an elcatonin 0.75 mU/kg/week administered group by Williams test p<0.05

##: comparison of nociceptive threshold of a vehicle administered group and an elcatonin 0.75 mU/kg/week

administered group by Williams test p<0.01

FIG. 2 shows serum calcium concentration after continuous administration of elcatonin for 14 days (after completion of the test in FIG. 1) on a sciatic nerve constriction model.

FIG. 3 shows withdrawal latency at the next day after initiation of administration in continuous administration of elcatonin for hyperalgesia of a sciatic nerve constriction model. A value before an operation is also shown for reference.

##: comparison of withdrawal latency of a vehicle administered group and an elcatonin administered group by Williams test p<0.01

FIG. 4 shows change in nociceptive threshold in intermittent administration of elcatonin using a sciatic nerve constriction model. An arrow shows a day on which elcatonin is subcutaneously administered intermittently.

**: comparison of nociceptive threshold before and after a constriction operation by t-test p<0.01

#: comparison of nociceptive threshold of a vehicle administered group and an elcatonin administered group by Williams test p<0.05

##: comparison of nociceptive threshold of a vehicle administered group and an elcatonin administered group by Williams test p<0.01

FIG. 5 shows change in serum calcium concentration in single subcutaneous administration of elcatonin in a normal rat.

### DETAILED DESCRIPTION OF PREFERRED EMBODIMENTS

The present invention will be illustrated specifically below.

As calcitonin useful as an active ingredient of the neuropathic pain treating and/or preventing agent of the present invention, various natural type calcitonins, or peptide analogues thereof and the like are mentioned. Examples of the natural type calcitonin include chicken calcitonin, eel calcitonin, human calcitonin, salmon calcitonin, pig calcitonin and the like, and eel calcitonin and salmon calcitonin (Helv. Chim. Acta (1969), 527), 1789-95) are mentioned as preferable examples, eel calcitonin is mentioned as particularly preferable example. In another embodiment, salmon calcitonin may be preferable.

As examples of peptide analogues of natural type calcitonin, mentioned are compounds obtained by chemically modifying a disulfide bond at 1,7-position based on a structure of the natural type calcitonin, and specifically, [ASU 1-7] chicken calcitonin, [ASU 1-7] eel calcitonin (chemical name: 1-butylic acid-7-(L-2-aminobutylic acid)-26-L-aspartic acid-27-L-valine-29-L-alaninecalcitonin described in Japanese Patent Application Publication (JP-B) No. 53-41677; hereinafter referred to also as “elcatonin” in some cases), and the like are mentioned as preferable examples, and [ASU 1-7] eel calcitonin (elcatonin) is mentioned as a particularly preferable example.

As calcitonin useful as an active ingredient of the agent for treating and/or preventing neuropathic pain of the present invention, elcatonin or salmon calcitonin is particularly preferable, and elcatonin is most preferable.

As the active ingredient of the agent for treating and/or preventing neuropathic pain of the present invention, salts of calcitonin useful as the active ingredient of the agent for treating and/or preventing neuropathic pain of the present invention, with pharmacologically acceptable acids maybe used. As the salts with pharmacologically acceptable acids, for example, salts with inorganic acids such as hydrochloric acid, sulfuric acid and the like, salts with organic acids such as acetic acid, tartaric acid, succinic acid, malic acid and the like are mentioned as suitable examples.

Further, a medicinal composition can also be produced using pharmacologically acceptable carriers according to an ordinary method.

Continuous administration of the present invention means an administration method of releasing a drug into a body continuously for a certain duration or more, and the administration route is not limited providing it is systemic administration or local administration to peripheral tissue, and for example, administration using a device such as an infusion pump, transfusion pump and the like or manual administration, and sustained-release preparations using as a carrier a polymer decomposed in a body, are mentioned. In contrast, intermittent administration means administering or releasing a drug into a body once or multiple times with an interval of certain time, not in continuous fashion.

The dosage form suitable for continuous administration of calcitonin includes a sustained release preparation, a drip preparation, a patch preparation and the like. The patch preparation includes also someone in which a fine needle is provided on a surface to be pasted to skin, and a drug oozes from the needle.

The continuous administration time in the present invention is preferably 8 hours or more, further preferably 12 hours or more, particularly preferably 16 hours or more.

Neuropathic pain means an abnormal condition of pain perception in which, due to a cause such as trauma, compression, infection, ischemia and the like, or metabolic disturbances such as diabetes and the like, neuropathy occurs in which nerve, plexus or perineural soft tissue is injured or degenerated, leading to continuation of lowering of pain threshold and the like due to some dysfunction caused by the neuropathy. Examples thereof include, but not limited to, spontaneous pain, pain threshold lowering (pain perception against mechanical stimulation), hyperalgesia (excess response to harmful stimulation) and hyperesthesia (excess response to contact, called also allodynia).

As diseases of neuropathic pain induced by a disorder of the peripheral nerve, neuropathic pains due to the compression of the peripheral nerve and injury of the peripheral nerve are mentioned, and particularly, diabetic neuropathy, entrapment (compression) peripheral neuropathy, spinal canal stenosis, disk herniation, carpal tunnel syndrome, reflex sympathetic dystrophy (RSD), shoulder-hand syndrome, CRPS (complex regional pain syndrome) type I, CRPS type II, postherpetic neuralgia, neuropathy due to HIV, atypical facial pain after exodontia and the like, trigeminal neuralgia and the like are mentioned. Further mentioned are postoperative pain, rheumatoid arthritis, osteoarthtiris and the like showing pain remaining also after administration of an anti-inflammatory analgesic.

Neuropathic pain caused by compression of the peripheral nerve means neuropathic pain induced by entrapment (compression) peripheral neuropathy, spinal canal stenosis, disk herniation, carpal tunnel syndrome, and the like.

The peripheral nerve means the sensory nerve, the motor nerve, the sympathetic nerve, the parasympathetic nerve, and the like, and the central nerve means the brain and the spinal cord nerve.

Neuropathic pain induced by peripheral neuropathy includes, but not limited to, diseases showing spontaneous pain, pain threshold decrease, hyperalgesia and allodynia in peripheral tissues. Further, neurogenic intermittent claudication, namely, augmentation of pain caused by addition of congestion load to peripheral neuropathy, is also included.

Neuropathy may be a mononeuropathy or polyneuropathy. Treatment effect is an effect by which neuropathic pain is treated by administering a drug after a nerve has been injured. More specifically, it is an effect of treating pain by returning the lowered pain threshold up to the normal value.

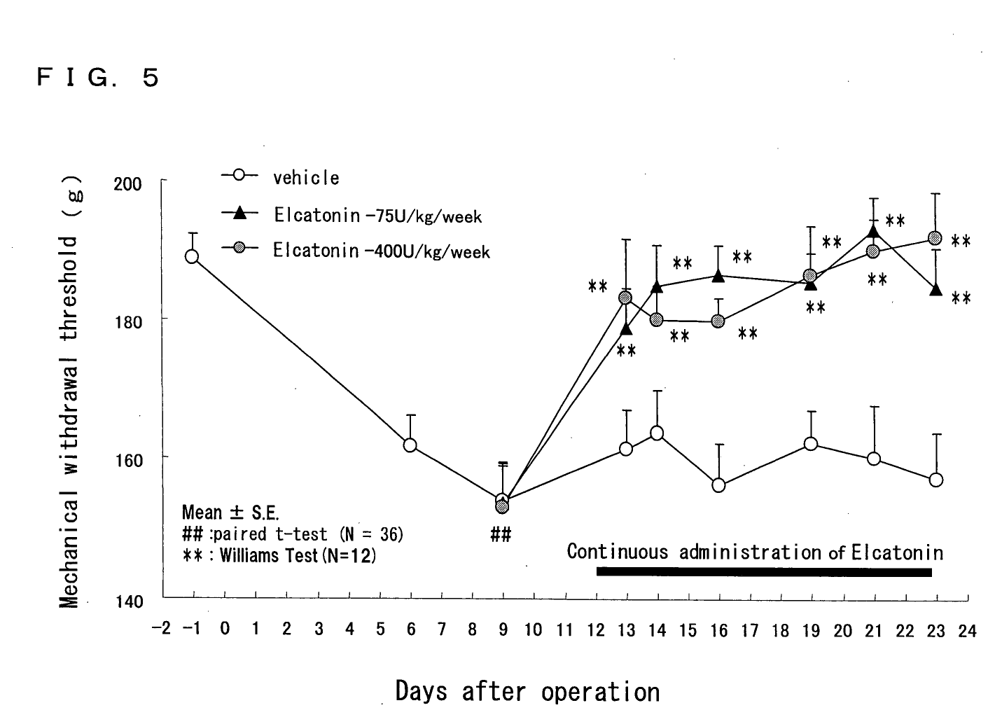

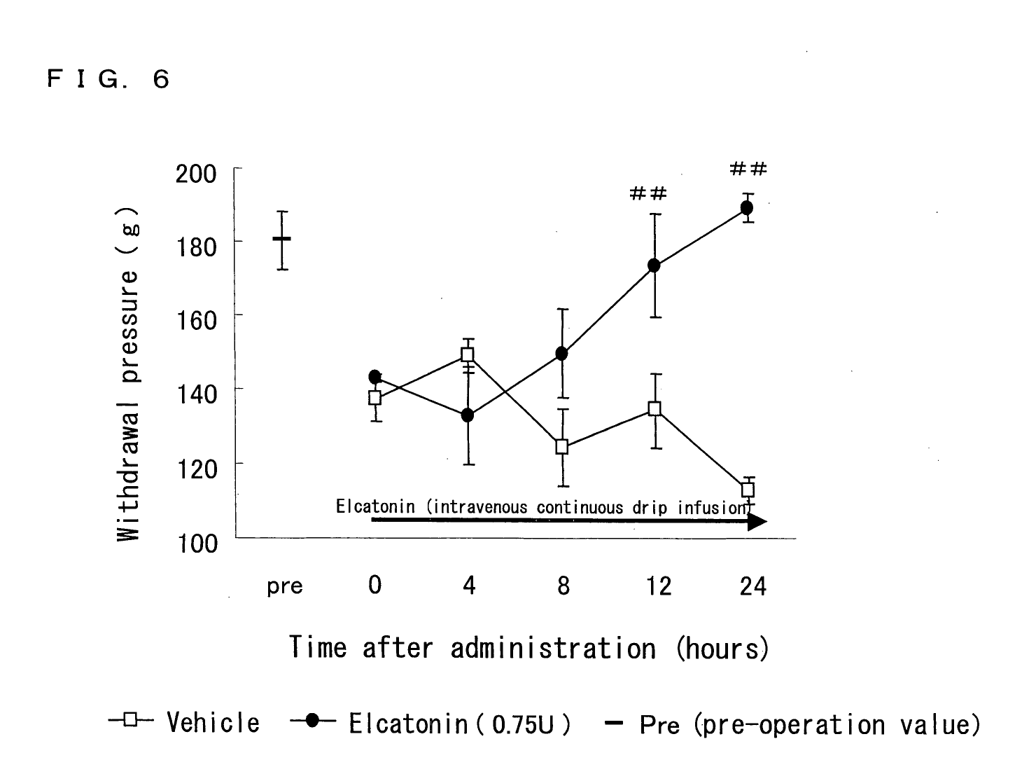

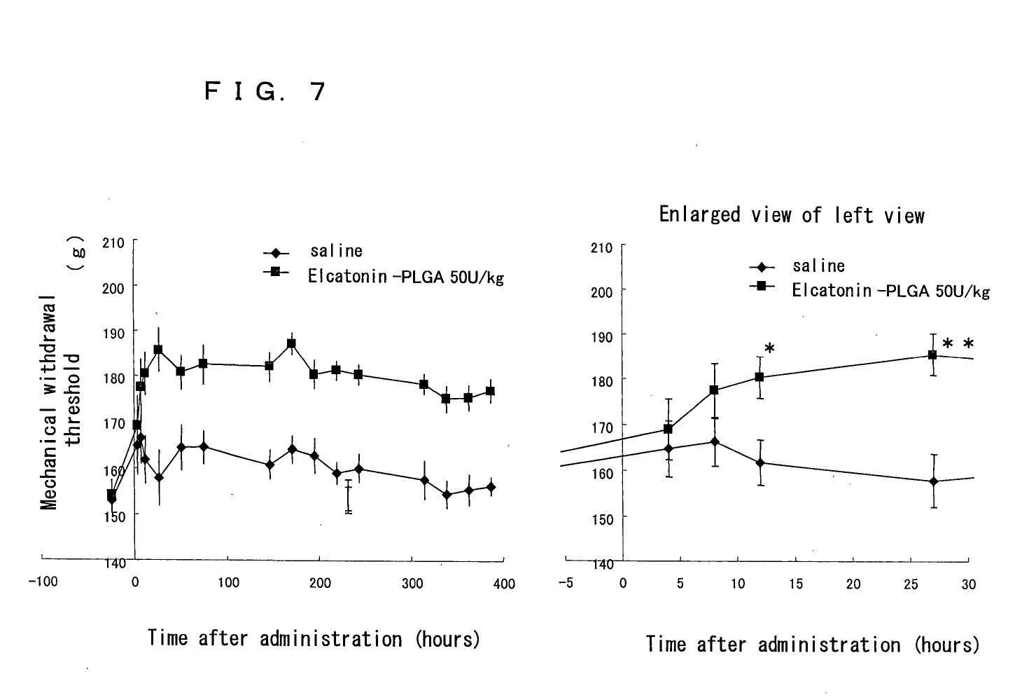

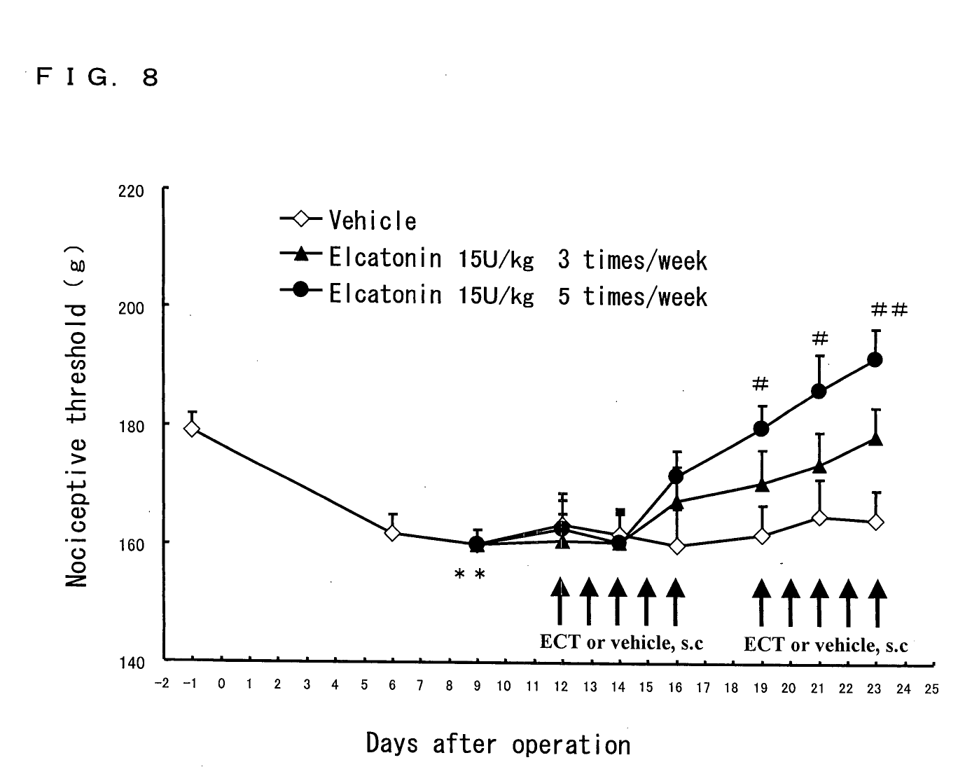

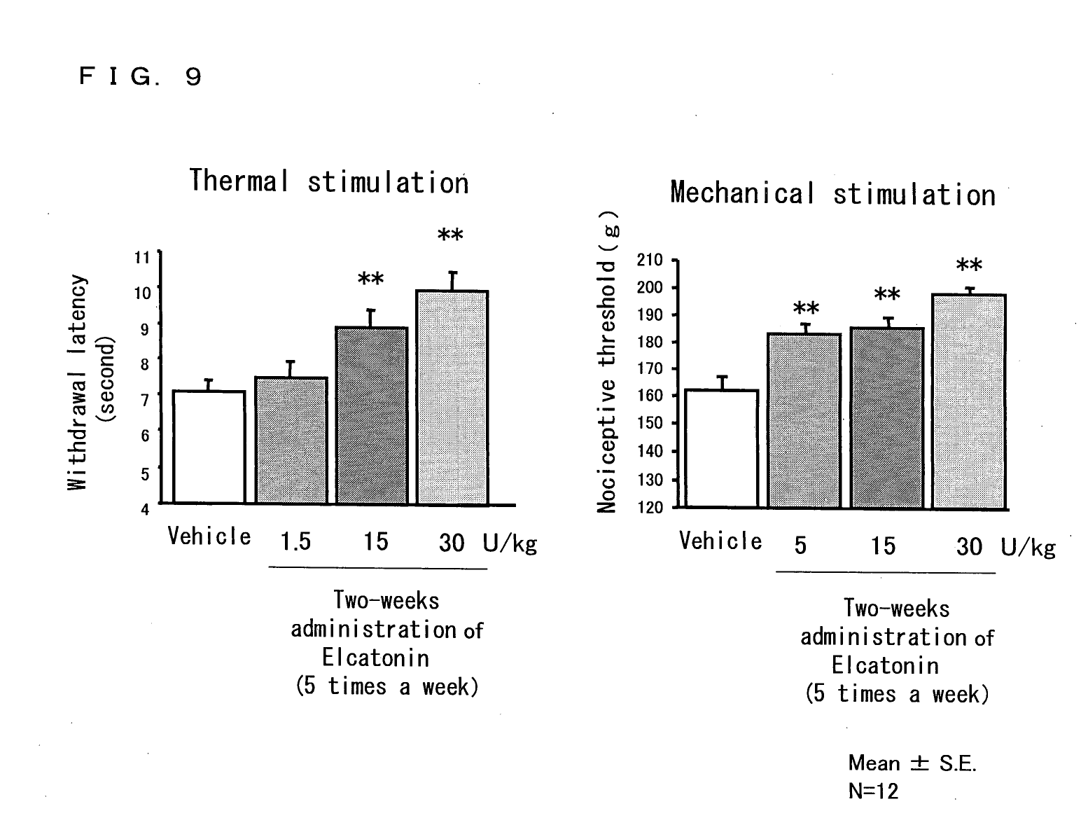

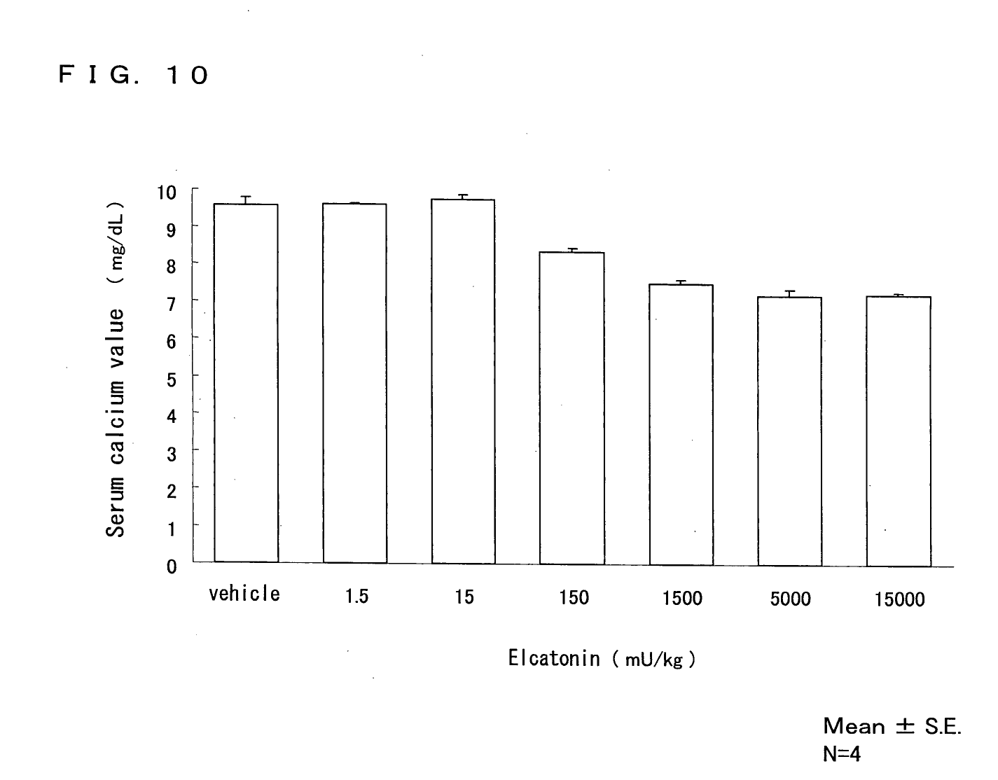

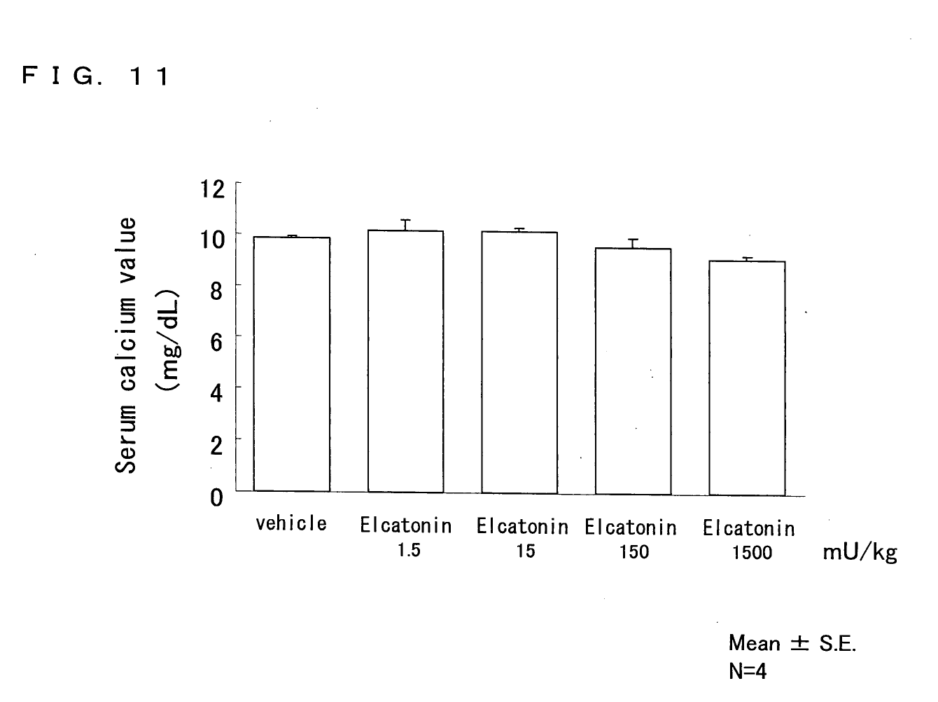

The amount of calcitonin sufficient for manifestation of an effect of treating neuropathic pain by continuous administration means an amount by which a neuropathic pain treatment effect is manifested by continuous administration to a patient or animal suffering from neuropathic pain. Preferably, it is a concentration that does not alter serum calcium values. As the dose of elcatonin that dose not alter serum calcium values in humans, less than 0.6 mU/kg/second are exemplified. Namely, test Example 1 described later checks the threshold of the elcatonin dose that does not alter serum calcium values in the case of single injection of elcatonin to a normal rat (subcutaneous injection over a period of 10 seconds), and as a result, serum calcium values change from the value in the case of administration of elcatonin 150 mU/kg, as shown in FIG. 5. In other words, the dose of 15 mU/kg/second is estimated to be the serum calcium alteration threshold. The calcitonin dose altering serum calcium value is estimated to be higher in rat by 25-fold than in human (Shinyaku to Rinsho, 1990, vol. 39, no. 2, p. 130-136). Therefore, it will be reasonable that the elcatonin dose altering serum calcium values in humans is one-25-th of that in rat, namely, 0.6 mU/kg/second. Thus, the continuous dose not altering serum calcium value in human is, for example, less than 0.6 mU/kg/second, preferably less than 0.09 mU/kg/second, further preferably less than 0.06 mU/kg/second.

The dose in the case of continuous administration for 1 week or more is preferably between 0.75 mU/kg/week to 75 U/kg/week as shown in Example 2 for rats. Further, the lower limit in human is preferably 0.003 mU/kg/week or more, more preferably 0.01 mU/kg/week or more, further preferably 0.03 mU/kg/week or more. The upper limit in humans is preferably less than 363 U/kg/week, more preferably 54 U/kg/week or less, further preferably 18 U/kg/week or less, most preferably 3 U/kg/week or less. The dose within this range results in approximately constant analgesia, and hypoalgesia caused by overdose does not occur, thus, dose variation of calcitonin is permissible in this range.

In another aspect of the present invention, it is preferable that the dose per unit time (second) of calcitonin in continuous administration is an amount equal to or less than one-hundredth of the dose per unit time (second) of calcitonin in each administration one shot effective for treatment of neuropathic pain in intermittent administration. Not bound by a theory, a dosage of 15 mU/kg/second is estimated to be the serum calcium-altering threshold, based on Test Example 1, FIG. 5, in single injunction of calcitonin to rats, as described above. The maximum effective dose of a neuropathic pain treatment by continuous administration in Example 2 is 75 U/kg/week 0.124 mU/kg/second, in terms of second). That is, 0.124 mU/kg/second is divided by 15 mU/kg/second to give a value of about one-hundredth. It is more preferably one-thousandth or less, further preferably one-five thousandth or less, most preferably one-fifteen thousandth or less. One-twenty thousandth or less are preferable in some embodiments.

Of course, specific dosage should be appropriately controlled depending on the body weight, the sex, the severity of symptoms and the like, and in no case should the patient's serum calcium values be altered.

A method of treating neuropathic pain, comprising a continuous administration for 8 hours or more of a calcitonin preparation in a dose of not lower than an amount necessary for effective therapy in neuropathic pain state and less than a dose that alters serum calcium values is also within the range of the present invention.

Examples of the dosage form of a medicine suitable in the present invention include sustained release preparations, for example, sustained release preparations by subcutaneous injection, intramuscular injection, intravenous injection or intraperitoneal injection, preferably, sustained release preparations by subcutaneous injection or intramuscular injection, drip preparations, patch preparations, and the like. As the sustained release preparations by subcutaneous injection, intramuscular injection, intravenous injection or intraperitoneal injection, exemplified are those prescribed so that calcitonin is used in the above-mentioned dosage, using microspheres using a biodegradable excipient such as poly(D,L-lactide-co-glycolide)polymer as disclosed in Japanese Patent Application National Publication (Laid-Open) No. 11-501027, for example. As the drip preparation, exemplified are those in which a medicine in preparations disclosed in “Nippon Rinsho, vol. 59, No. 9, p. 1789-1793, 2001” is replaced by calcitonin and prescription is so made as to give the above-mentioned dose of calcitonin. The patch preparation includes a patch preparation of type in which a fine needle is provided on a surface to be pasted to skin, and a drug oozes from the needle, and for example, exemplified are those patch preparations disclosed in Japanese Patent Application National Publication (Laid-Open) No. 2004-528900 in which prescription is so made as to give the above-mentioned dose of calcitonin. Needless to say, the preparation is not limited to these examples.

In addition, exemplified as the sustained release preparation is that obtained by filling a mini osmotic pump (2002) manufactured by ALZA with calcitonin so as to give the above-mentioned dosage amount as shown in Example 1 described later, and this can be implanted subcutaneously at neck.

The continuous administration of the present invention may be simultaneously used with the intermittent administration of calcitonin or an osteoporosis therapeutic agent for a patient or animal with both osteoporosis and neuropathic pain.

Further, use of calcitonin for producing an agent for treating and/or preventing neuropathic pain is also within the range of the present invention.

### EXAMPLES

The present invention will be illustrated based on examples and reference examples, but the range of the present invention is not limited to the following examples.

### Example 1

For confirming an effect of continuous administration of elcatonin (ECT) in neuropathic pain by peripheral nerve injury, a chronic constriction injury model (CCI model) described in Namiki et al., Masuikai to Kisokenkyu: Totsu to Chintsu (NANKO DO), p. 37 (2000) as its model animal was improved and studied. Four-week old male SD (IGS) rats was purchased and subjected to preliminary breeding, taming by handling, and domestication for a measuring apparatus of mechanical withdrawal threshold, each for 5 days, then, at 7-weeks old, a sciatic nerve constriction operation or a sham-operation was carried out and the rats were tested.

Under anesthesia with ether, a rat was placed in prone position, and while touching the right femur, the skin was dissected right above it. Biceps femoris was peeled at the center of the femur, and exposed by about 5 mm trying not to injure the sciatic nerve. Using 4-0 blade silk, the sciatic nerve was liegted loosely 4 times in turn from the peripheral side. Thereafter, the fascia and the skin were sutured.

Using withdrawal threshold and body weight on the 8th day after the constriction operation as indices, the rats were divided into a CCI-vehicle group (12 rats), and CCI-ECT 0.75 mU/kg/week, CCI-ECT 7.5 mU/kg/week and CCI-ECT 75 mU/kg/week groups (each 12 rats), by a randomization method (SAS system, version 8.2). On the 11th day after the constriction operation, amini osmotic pump (2002 model) manufactured by ALZA was filled with a drug to attain the above-mentioned administration amounts, and implaned subcutaneously at neck under ether anesthetization. As a vehicle, 0.02% BSA-containing 0.1 mM sodium acetate buffer (pH 5.5) was used. The flow rate of the mini osmotic pump manufactured by ALZA was constant 0.5 μL/hour), and the dose of elcatonin was controlled by the concentration of elcatonin to be filled. Body weight used for administration was estimated body weight after initiation of administration and calculated by multiplying body weight at start of administration by body weight increase coefficient (1.1) obtained in the preliminary experiment. The concentration of filled elcatonin was confirmed by HPLC or sandwich type ELISA in which two polyclonal antibodies labeled with beta-galactosidase are bonded to an antigen (elcatonin) and measured.

Measurement (Randall Selitto method) by an analgesic effect measuring apparatus against mechanical stimulation was carried out according to a manual of Analgesy meter (UGO BASILE: 7200). Immediately after initiation of administration, the position of a cage was moved randomly using random number by a person other than a withdrawal latency measuring staff, further, an identification seal on the cage was peeled, thus, measurement was carried out under condition wherein the measuring staff could not identify the individual rats.

First, the position of a pressure needle was controlled to confirm that an arm was horizontal to the thickness of a rat leg. A rat right posterior limb was placed between a pedestal and a pressure needle at the arm end, then, a moving matter was set at 0, a foot pedal was trodden, and the screw rod was rotated and the moving matter was moved at a rate of 16 mm/second, and a load (16 g/second) was applied to the pressure needle. When the rat withdrew the limb, the foot pedal was released, and a numerical value 0 to 25) of the moving matter on a scale was read. The read numerical value was multiplied by 20, to convert into pressed weight (withdrawal threshold). The average value of two measurement values was used as the withdrawal threshold of an individual rat.

In statistical analysis of the result, for confirmation of lowering the of threshold in the CCI vehicle administration group, a value before the operation and the threshold on 9th day after the operation were checked by the paired t-test (SAS system, version 8.2), and a significance of 5% or less was considered to be statistically different (** in a figure: p<0.01). Comparison of the CCI vehicle administration group and the CCI ECT administration group was analyzed by Williams test, and a hazard ratio of 2.5% or less was judged to be significant (only CCI ECT 0.75 mU/kg group is shown in a figure, #: p<0.05, ##: p<0.01).

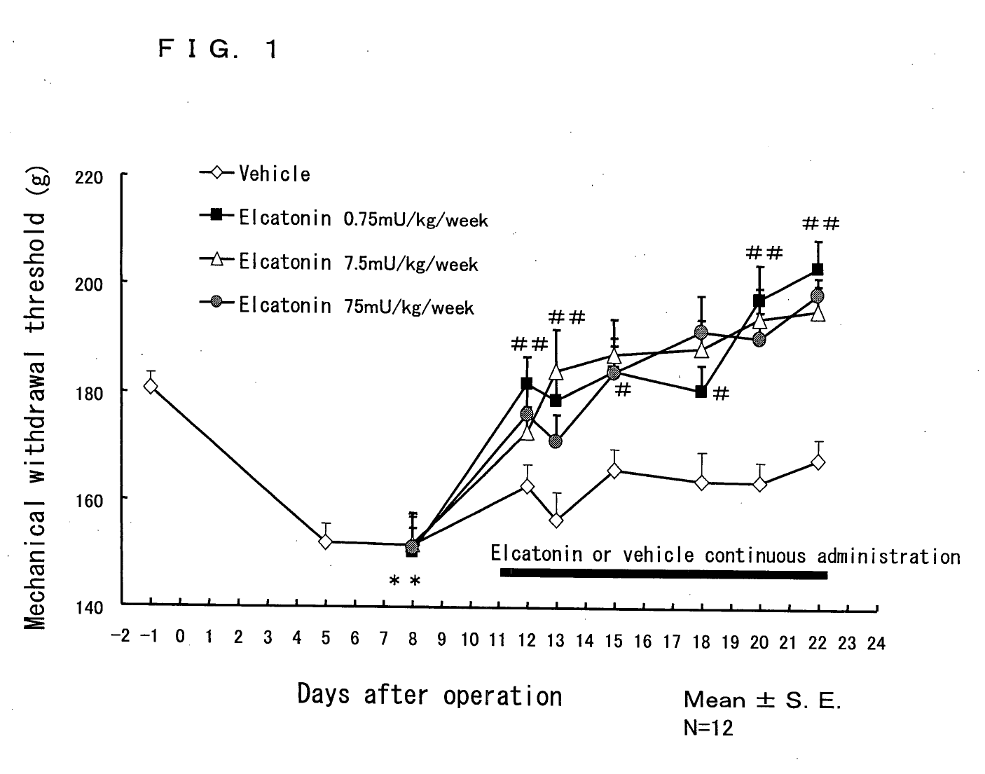

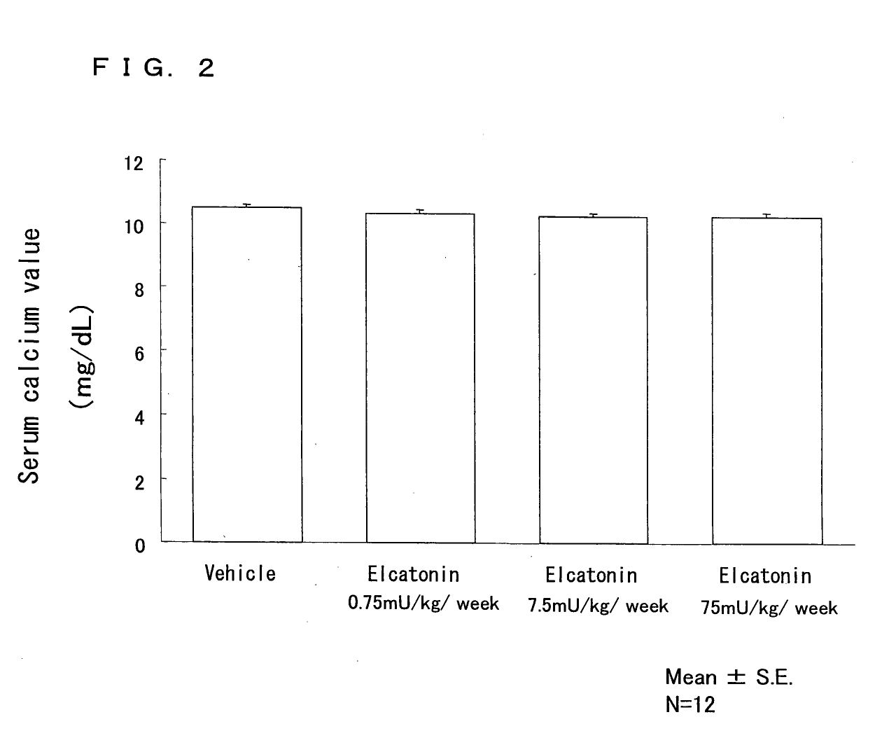

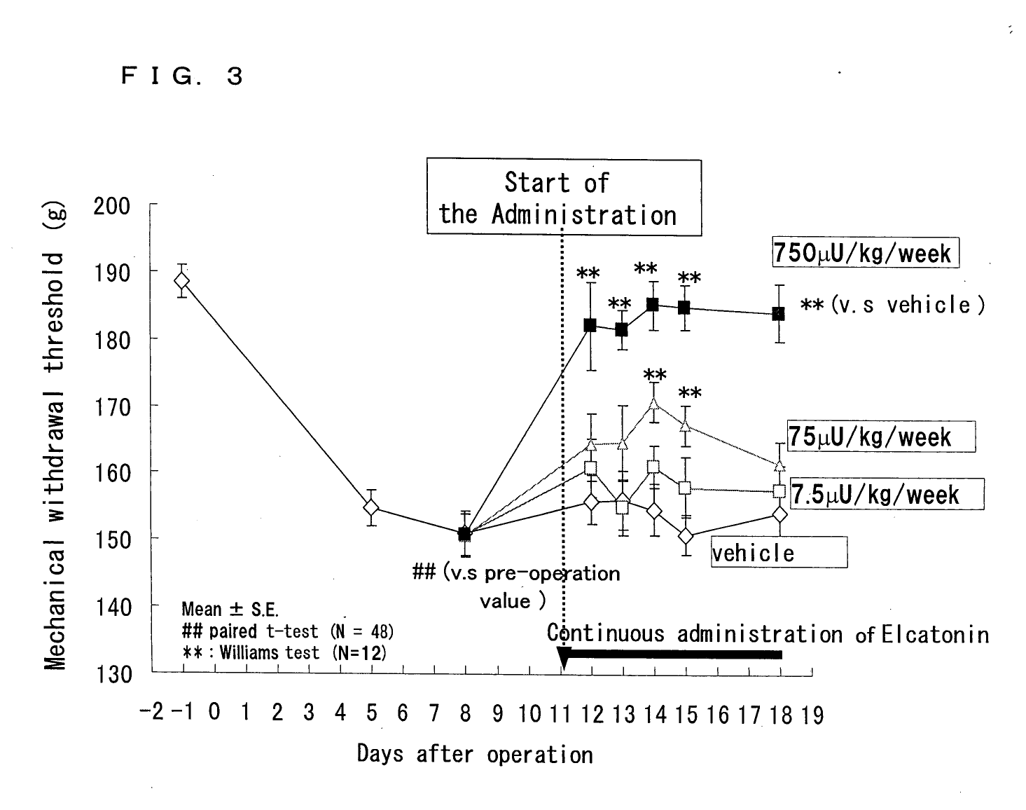

The results are shown in FIG. 1. A significant analgesic action was recognized from the next day after the initiation of the administration (16 hours after), and continued to completion of the test. Further, on 14th day after the initiation of the administration, serum calcium concentration was measured, as a result, as shown in FIG. 2, elcatonin continuous administration did not affect serum calcium amount at all. In the elcatonin continuous administration group, no change in body weight was found as compared with the vehicle administration group.

Therefore, it was clarified that elcatonin continuous administration shows a quick-acting excellent analgesic action for lowering of the nociceptive threshold of a neuropathic pain model.

### Example 2

Using the same CCI model as in Example 1, an effect of elcatonin continuous administration on hyperalgesia occurring in the CCI model was investigated. Preparing of the CCI model, elcatonin administration method and administration initiation period were the same as in Example 1. The elcatonin dose was 0.00075 U/kg/week, 0.0075 U/kg/week, 0.075 U/kg/week, 7.5 U/kg/week or 75 U/kg/week, and a vehicle was administered in the CCI control group. Measurement was carried out according to a manual of “BASILE Plantar Test” (UGO BASILE: 7370). An apparatus measures a time (withdrawal latency) until escaping after giving nociceptive heat stimulus to a posterior limb in a rat under unrestrained condition. Right hindlimb was measured as a measurement leg. Initially, a rat was placed in an acryl box on a glass plate, and domesticated for about 5 minutes. Amoving type I.R (infrared) generator (I.R. vessel) was placed under the glass plate, and “sight” cross (I.R. irradiation position) inscribed on an upper panel of the I.R. vessel was adjusted to inside of six pads on the right hind paw. In this procedure, contact of the glass plate and hind paw was confirmed. Subsequently, a start key was pushed and heat stimulation was imparted, and withdrawal latency for escaping behavior of a rat of withdrawing limb was measured. When escaping behavior is caused, a switch is automatically turned off, and reaction time is counted. In this measurement, stimulation of I.R. strength of 80 was used, and cut-off time was 22.5 seconds (regulated value). Measurement was carried out under clean condition so that there was no feces and no urine on the glass plate. The mean of two measurement values was used as the withdrawal latency of an individual rat.

In statistical analysis of the result, for confirmation of hyperalgesia (shortening of withdrawal latency) in the CCI vehicle administration group, a value before the operation and the threshold on 9th day after the operation were checked by the paired t-test (SAS system, version 8.2), and a significance of 5% or less was considered to be significant. Comparison of the CCI vehicle administration group and the CCI ECT administration group was analyzed by Williams test, and a significance of 2.5% or less was considered to be significant (shown in a figure, ##: p<0.01).

The withdrawal latency on the next day after the initiation of the administration (on 12th day after operation) was shown in FIG. 3. A value before the operation is also shown as a reference in FIG. 3. A significant analgesic action was confirmed in all elcatonin administration groups as compared with the vehicle group. Administration was continued for 2 ensuring weeks, to find continuance of an analgesic action.

Further, on 14th day after initiation of administration, serum calcium concentration was measured, to find that elcatonin continuous administration did not affect serum calcium amount at all.

Therefore, it was clarified that a quick acting excellent analgesic action is shown also on hyperalgesia of a neuropathic pain model. Further, in these dose ranges, extent of an analgesic action was approximately constant, and hypoalgesia over a normal value did not occur.

### Comparative Example

An action of intermittent administration of elcatonin was investigated in the same manner as in Example 1.

From two weeks after in coming until initiation of elcatonin (ECT) administration, a vehicle 0.02% BSA-containing 0.1 mM sodium acetate buffer (pH 5.5)) was administered subcutaneously 5 times a week at 0.05 ml/B.W.100 g to all animals. Using withdrawal threshold and body weight on 9th day after the constriction operation as indices, rats were distributed into a CCI-vehicle group (12 individuals), and CCI-ECT 15 U/kg/3 times a week (Monday, Wednesday, Friday; vehicle for Tuesday and Thursday) and CCI-ECT 15 U/kg/5 times a week (Monday, Tuesday, Wednesday, Thursday, Friday) groups (each 12 rats), by a randomization method (SAS system, version 8.2). On the 12th day or later after the constriction operation, ECT or vehicle was administered subcutaneously at 0.05 ml/B.W.100 g.

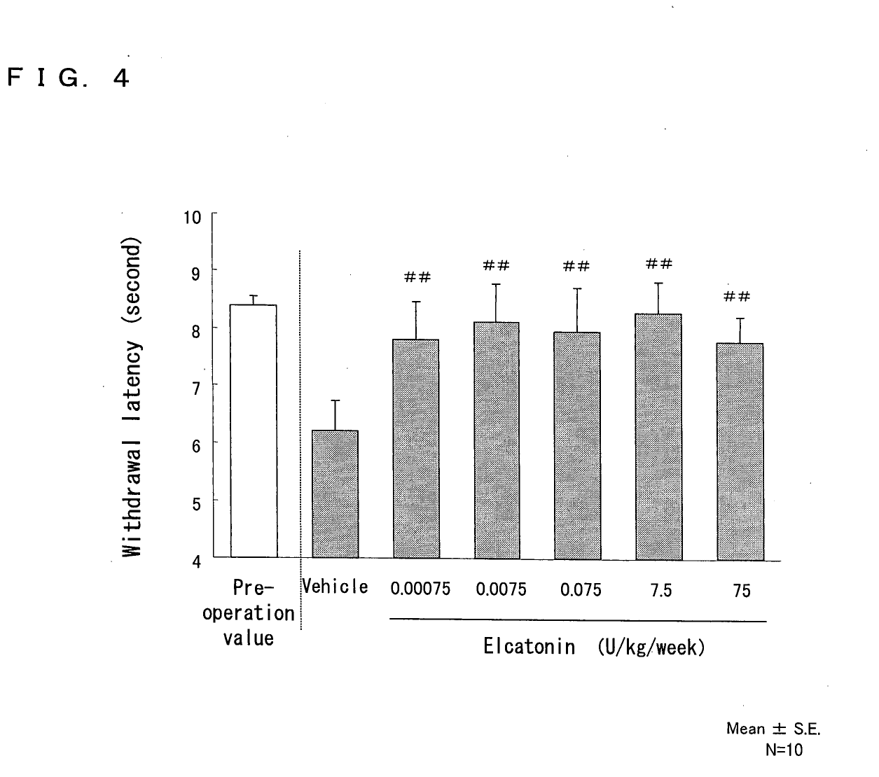

The result is shown in FIG. 4. In the CCI model, a significant lowering of nociceptive threshold was recognized as compared with that before the operation (**: p<0.01). Until 3-rd day after initiation of ECT administration, an effect was not observed, while in the ECT 5 times a week administration group, from 4-th day after initiation of administration, the threshold initiated to recover gently, and on 7th day or later, a significant analgesia action was recognized (#: p<0.05, ##: p<0.01). In the ECT 3 times a week administration group, an improvement tendency was observed, however, a significant action was not observed. In the ECT administration group, suppression of body weight increase was recognized. Separately, the dose was further increased and a test was conducted, as a result, delay of manifestation of an analgesic effect was not improved, and suppression of body weight increase became remarkable, thus, it was considered that this is not suitable as a therapeutic medicine.

Therefore, it could be confirmed that elcatonin intermittent administration improves neuropathic pain, but its efficiency is delayed and a satisfactory treatment effect is not obtained. Both in the CCI-ECT 15 U/kg 3 times a week administration group and the CCI-ECT 15 U/kg 5 times a week administration group, variation in serum calcium value was observed.

### Test Example 1

Using normal rats (each 4 rats) which were in the same week old in initiation of administration of elcatonin as in Examples 1 and 2, dose of elcatonin not varying serum calcium value was examed in the case of single subcutaneous administration (administered over 10 seconds). Timing of blood collection was 30 minutes after the administration at which serum calcium value of a rat by elcatonin was maximum. Measurement of serum calcium value was conducted using calcium C-test wako (Wako). As shown in FIG. 5, serum calcium value were changed at 150 mU/kg or more, namely, at 15 mU/kg/second or more per unit time (second), in single subcutaneous injection.

From the above-mentioned results, it was confirmed that a calcitonin continuous administration preparation in neuropathic pain is a useful agent for treatment and/or prevention, which manifests an excellent action even at lower dosage as compared with intermittent administration, and can reduce side effects and dosage frequency.

### INDUSTRIAL APPLICABILITY

The present invention shows an effective action on neuropathic pain and is suitable as a medicine.

1. A method of treating and/or preventing neuropathic pain, comprising administering to a patient in need of a method of treating and/or preventing neuropathic pain calcitonin in an amount not altering serum calcium values of the patient and in an amount effective for the treatment and/or prevention of neuropathic pain.

2. The method of treating and/or preventing neuropathic pain according to claim 1, wherein the patient is a human.

3. The method of treating and/or preventing neuropathic pain according to claim 1, comprising administering calcitonin in an amount less than 0.093 mU/kg/second.

4. The method of treating and/or preventing neuropathic pain according to claim 1, wherein the administration is a continuous administration.

5. The method of treating and/or preventing neuropathic pain according to claim 4, wherein the dose per unit time (second) of calcitonin in continuous administration is an amount equal to or less than one-fourth of the serum calcium alteration threshold dose per unit time (second) of calcitonin.

6. The method of treating and/or preventing neuropathic pain according to claim 3 or 4, wherein the duration of the continuous administration is 8 hours or more.

7. The method of treating and/or preventing neuropathic pain according to claim 1, wherein the neuropathic pain is caused by the compression of the peripheral nerve or the injury of the peripheral nerve.

8. An agent for treating and/or preventing neuropathic pain comprising calcitonin as an active ingredient, wherein calcitonin is administered in an amount not altering serum calcium values.

9. An agent for treating and/or preventing neuropathic pain comprising calcitonin as an active ingredient, wherein calcitonin is administered in an amount less than 0.093 mU/kg/second.

10. The agent for treating and/or preventing neuropathic pain according to claim 8, wherein the agent is administered continuously.

11. The agent for treating and/or preventing neuropathic pain according to claim 10, wherein the dose per unit time (second) or calcitonin in continuous administration is an amount equal to or less than one-fourth of the serum calcium alteration threshold dose per unit time (second) of calcitonin.

12. The agent for treating and/or preventing neuropathic pain according to claim 10, wherein the duration of the continuous administration is 8 hours or more.

13. The agent for treating and/or preventing neuropathic pain according to claim 8, wherein the neuropathic pain is caused by the compression of the peripheral nerve or the injury of the peripheral nerve.

14. The agent for treating and/or preventing neuropathic pain according to claim 8, wherein the calcitonin is elcatonin.

15. An agent for treating and/or preventing neuropathic pain for continuous administration comprising calcitonin as an active ingredient, wherein the dosage form may be a sustained release preparation, a drip preparation or a patch preparation.

16. The agent for treating and/or preventing neuropathic pain according to claim 15, wherein the dosage form may be an injection preparation, an implanted preparation, a percutaneous preparation, a transnasal preparation, a transpulmonary preparation or a peroral preparation.

17. The agent for treating and/or preventing neuropathic pain according to claim 15, wherein the dose per unit time (second) of calcitonin in continuous administration is an amount equal to or less than one-fourth of the serum calcium alteration threshold dose per unit time (second) of calcitonin.

18. The agent for treating and/or preventing neuropathic pain according to claim 15, wherein the duration of the continuous administration is 8 hours or more.

19. Use of calcitonin for producing the agent for treating and/or preventing neuropathic pain according to claim 8 or 15.
